(19) United States
(12) Patent Application Publication
(10) Pub. No.: US 2006/0210543 A1
(43) Pub. Date: Sep. 21, 2006

(54) Method of treating infarcted myocardium

(76) Inventors: Jonathan Leor, Ganei Tikva (IL); David Danon, Ganei Tikva (IL)

Correspondence Address: PRTSI, Inc., P.O. Box 16446, Arlington, VA, 22215, US

(21) Appl. No.: 11/384,376

(22) Filed: Mar. 21, 2006

Related U.S. Application Data
(60) Provisional application No. 60/663,268, filed on Mar. 21, 2005.

(51) Int. Cl. A61K 35/14

(52) U.S. Cl. 424/93.7

(57) ABSTRACT

A method of treating an infarcted myocardium, the method comprising administering to the myocardium of a subject in need thereof a therapeutically effective amount of osmotically activated immune cells, thereby treating the infarcted myocardium.

### RELATED APPLICATIONS

This application claims priority from U.S. Provisional Patent Application No. 60/663,268, filed on Mar. 21, 2005, the contents of which are incorporated herein by reference.

### FIELD AND BACKGROUND OF THE INVENTION

The present invention relates to a method of treating infarcted myocardium using osmotically activated immune cells.

Myocardial infarction (MI) is characterized by the death of myocytes, coagulative necrosis, myocytolysis, contraction band necrosis, or apoptosis, resulting from a critical imbalance between the oxygen supply and demand of the myocardium. The most common cause of MI is coronary artery thrombosis following the rupture of atheromatous plaques. Though once strictly defmed as a lack of blood flow, the modern definition of ischemia emphasizes the imbalance between oxygen supply and demand as well as the inadequate removal of metabolic waste products. Impaired oxygen delivery results in a reduction in oxidative phosphorylation that causes anaerobic glycolysis. This produces excess lactate that accumulates in the myocardium. Impaired ATP production and acidosis results in a decline in myocardial contractility. Similarly, ischemia reperfusion injury, without total occlusion, can also cause cardiac damage. The exposure of the contents of the plaque to the basement membrane following plaque rupture ultimately results in vessel blockage culminating from a series of events including platelet aggregation, thrombus formation, fibrin accumulation, and vasospasm. Total occlusion of the vessel for more than 4-6 hours results in irreversible myocardial necrosis. Ultimately, death and morbidity from myocardial infarction is the result of fatal dysrhythmia or progressive heart failure. Progressive heart failure is chiefly the result of insufficient muscle mass (deficiency in muscle cells) or improper function of the heart muscle, which can be caused by various conditions including, but not limited to, hypertension. Progressive heart failure is, therefore, the focus of cell-based therapy.

All current strategies for the treatment of myocardial infarction (MI) focus on limiting myocyte death. Annually in the United States, 500,000 patients undergo angioplasty with stent placement. 400,000 will undergo coronary artery bypass, while an unknown additional number of patients will be treated by thrombolytic therapy.

The inflammatory response following MI is determinative for tissue healing (Frangogiannis et al., 2002, Cardiovasc Res 53, 31-47). To date, reperfusion is the preferred clinical therapy for MI and is associated with higher presence of inflammatory cells, particularly macrophages and leukocytes, enhanced neovascularization, and less adverse remodeling (Vandervelde et al., 2006 Cardiovasc Pathol 15, 83-90).

One approach, known as cellular cardiomyoplasty, has received recent attention and focuses on repopulation and engraftment of the injured myocardium by transplantation of healthy cells [Reffelmann, T. and Kloner, R. A. (2003) Cardiovasc Res. 58 (2): 358-68]. Many cell types that might replace necrotic tissue and minimize regional scarring have been considered. Cells that have already committed to a specific lineage, such as satellite cells, cardiomyocytes, primary myocardial cell cultures, fibroblasts, and skeletal myoblasts, have been readily used in cellular cardiomyoplasty with limited success in restoring damaged tissue and improving cardiac function [Etzion, S. et al (2001) J Mol Cell Cardiol. 33 (7)].

Cardiogenic progenitors are precursor cells that have committed to the cardiac lineage, but have not differentiated into cardiac muscle. Cardiomyocytes are the cells that comprise the heart. They are also known as cardiac muscle cells. Use of cardiomyocytes in the repair of cardiac tissue has been proposed. However, this approach is hindered by an inability to obtain sufficient quantities of cardiomyocytes for the repair of large areas of infarcted myocardium. Doubt has also been cast over the incorporation and tissue-specific function of intra-cardiac grafts derived from cardiomyocytes, even when they are harvested from embryonic sources [Etzion, S. et al (2001) J. Mol. Cell. Cardiol. 33 (7): 1321-30]. Intra-cardiac grafts using this cell type can be successfully grafted and are able to survive in the myocardium after permanent coronary artery occlusion and extensive infarction. However, engrafted rat embryonic cardiomyocytes attenuate, but do not fully reverse left ventricular dilatation and prevent wall thinning. While survival was improved during 8 weeks of follow-up, the implanted cells did not develop into fully differentiated myocardium. Surprisingly, they remained isolated from the host myocardium by scar tissue and did not improve systolic finction over time (Etzion, S. et al (2001) J. Mol. Cell. Cardiol. 33 (7): 1321-30). There is thus a widely recognized need for a novel cell therapy approach for treating an infarcted myocardium which is devoid of the above limitations.

U.S. Pat. Publ. No. 20050129663 teaches the use of dermis activated macrophages for axonal regeneration in the CNS, wound healing and treatment of myocardial infarction. However, this approach is laborious in necessitating the isolation of skin segments from the patient and co-incubating of same with the white blood cell sample. Additionally, the white blood cell sample is prone to contamination arising from the skin segments and therefore cannot be favored as a therapeutic modality. As expected, no clinical results using this approach for treating infarcted myocardium were reported.

GM-CSF stimulated macrophages. were used for augmenting collateral vessel growth in an animal model of arteriogenesis (Herold 2004) but not for treating myocardial infarction which treatment requires also the formation of. extracellular matrix in-order to repair and regenerate a functional tissue.

The present inventors have previously used human hypo-osmotically activated macrophage suspensions for the treatment of unhealed ulcers and wounds by local injections [Danon Exp Gerontol. 1997;32:633-41; Zuloff-Shani Transfus Apheresis Sci. 2004;30: 163-7]. However, use of osmotically activated macrophages suspensions for the treatment of infarcted myocardium has never been suggested to date.

### SUMMARY OF THE INVENTION

According to one aspect of the present invention there is provided a method of treating an infarcted myocardium, the method comprising administering to the myocardium of a subject in need thereof a therapeutically effective amount of osmotically activated immune cells, thereby treating the infarcted myocardium.

According to further features in preferred embodiments of the invention described below, the administering comprises local administering.

According to still further features in the described preferred embodiments the local administering is effected by injection.

According to still further features in the described preferred embodiments the method further comprising activating white blood cells so as to obtain the osmotically activated immune cells prior to the administering.

According to still further features in the described preferred embodiments the activating is effected by subjecting the white blood cells to a hypotonic solution.

According to still further features in the described preferred embodiments the hypotonic solution is distilled water.

According to still further features in the described preferred embodiments the osmotically activated immune cells are administered at an amount selected from 0.1-10×10⁶ cells/Kg body weight.

According to another aspect of the present invention there is provided an article of manufacturing comprising packaging material and a pharmaceutical composition identified for treating an infarcted myocardium being contained within the packaging material, the pharmaceutical composition comprising, as an active ingredient, osmotically activated immune cells and a pharmaceutically acceptable carrier.

According to still further features in the described preferred embodiments pharmaceutical composition is formulated for local administration.

According to still further features in the described preferred embodiments the infarcted myocardium is associated with a disease or condition selected from the group consisting of atherosclerosis, ventricular hypertrophy, hypoxia, emboli to coronary arteries, coronary artery vasospasm, arteritis, coronary anomaly.

According to still further features in the described preferred embodiments the osmotically activated immune cells comprise macrophages.

According to still further features in the described preferred embodiments the osmotically activated immune cells comprise non-autologous cells.

According to still further features in the described preferred embodiments the non-autologous cells comprise xenogeneic cells.

According to still further features in the described preferred embodiments the non-autologous cells comprise allogeneic cells.

According to still further features in the described preferred embodiments the osmotically activated immune cells comprise hypo-osmotically activated immune cells.

The present invention successfully addresses the shortcomings of the presently known configurations by providing a method of treating an infarcted myocardium which is devoid of prior art limitations.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-d are MRI photographs which track human AMS cells to the place of administration, left ventriculat anterior wall. AMS cells were labeled with magnetic resonance (MR) iron oxide nanoparticle solution together with the transfection agent Poly-L-Lysine (photographs of treated hearts are labeled Fe-PLL AMS). The chest area was scanned using the 0.5T GE iMRI machine with a specially constructed animal probe, 1 (FIGS. 1a, c) and 8 (FIGS. 1b, d) days following injections, as indicated. Imaging sequences showed strong black signal (arrows) from the left ventricular (LV) anterior wall of AMS-treated hearts (FIGS. 1a-b) but not in controls (FIGS. 1c-d).

FIGS. 2a-b are photographs showing presence of human growth hormone (HGHof human AMS cells in the infarcted rat heart. DNA was extracted following AMS injection. AMS cell presence was detected 2 (FIG. 2a), 4 and 7 (FIG. 2b) days following injection, using PCR for human growth hormone (GH) gene (The arrows indicate the position of the expected 434 bp products). FIG. 2a—lane A, PCR template was AMS DNA (positive control); lanes B and C, PCR templates were DNA from different AMS-treated hearts, and a positive signal is shown in both cases. In lane D PCR template was DNA from saline treated heart (negative control), and no signal is shown. FIG. 2b—lanes A-B, PCR template was DNA from 2 different AMS -treated hearts, 4 days after injection. A weak positive signal is indicated by a circle. In lanes C-D, PCR template was DNA from two individual AMS- treated hearts 7 days following injection. A very weak positive signal (lane C) or no signal (lane D) was obtained. In lanes E-F, PCR template was DNA from saline-treated hearts 4 days after injection (negative control). In lane G, PCR template was AMS DNA (positive control). In lane H, PCR was conducted with no DNA template (negative control).

FIGS. 3a-b are photomicrographs depicting the detection of human cells in rat hearts 4 days following treatment, by CD68 immunohistochemistry. A section from AMS-treated hearts was stained with an antibody to monocyte / macrophage antigen —CD68. Staining shows a few aggregates of positive brown staining in AMS treated hearts (FIG. 3a), as compared to the control section (FIG. 3b) of an untreated heart that shows no staining. Original magnification x 400.

FIGS. 4a-b are photomicrographs depicting higher presence of rat tissue-resident monocytes and macrophages, two months after AMS injection (FIG. 4a), as compared to untreated hearts (FIG. 4b). Sections were immunostained with EDI, and co-stained with hematoxylin, a marker for rat tissue-resident macrophages, 8 weeks after transplantation. AMS-treated scars exhibited greater accumulation of resident macrophages (brown cells) associated with intensive vascularization (v), compared with controls (original magnification ×200).

FIGS. 5a-d are photomicrographs depicting α-SMA immunostaining of scar tissue. Scar tissue sections of AMS treated (FIGS. 5a,c) and control tissue (FIGS. 5b, d), were immunostained with Smooth muscle α-actin. When compared to controls, AMS implantation promoted scar tissue vascularization (upper panel). The red blood cells (arrows) in the vessel lumens indicate functional vessels. Activated macrophage injection also promoted myofibroblasts accumulation in the scar tissue, as indicated by dense brown staining in the lower panel (original magnification ×200).

FIGS. 6a-f are bar graphs depicting improved LV remodeling and function of AMS treated hearts, when compared to contols, as shown by a 2D echocardiography study, before (baseline) and two months after injection. Compared with controls (white bars), AMS (red bars) improved scar thickening (FIG. 6a); attenuated LV diastolic dilatation (FIG. 6b); LV systolic dilatation (FIG. 6c); LV diastolic area (FIG. 6d); LV systolic area (FIG. 6e); and improved LV fractional shortening (FIG. 6f). At two months after injection, all differences between groups are significant (p<0.05).

### DESCRIPTION OF THE PREFERRED EMBODIMENTS

The present invention is of a method of treating infarcted myocardium using osmotically activated immune cells.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Myocardial infarction (MI) is characterized by the death of myocytes, coagulative necrosis, myocytolysis, contraction band necrosis, or apoptosis, resulting from a critical imbalance between the oxygen supply and demand of the myocardium. The most common cause of MI is coronary artery thrombosis following the rupture of atheromatous plaques.

Current approaches for treating MI focus on limiting myocyte death such as by angioplasty using thrombolytic therapy, stent placement and coronary artery bypass.

Cell therapy for the treatment of MI has received recent attention and focuses on repopulation and engraftment of the injured myocardium by transplantation of healthy cells [Reffelmann, T. and Kloner, R. A. (2003) Cardiovasc Res. 58 (2): 358-68]. Committed cells such as fibroblasts and skeletal myoblasts, have been readily used in cellular cardiomyoplasty with limited success in restoring damaged tissue and improving cardiac function. Alternatively, the use of cardiogenic progenitors and stem cells is limited by insufficient quantities of such cells for the repair of large areas of infarcted myocardium as well as by poor efficacy.

There is thus a widely recognized need for a novel cell therapy approach for treating the infarcted myocardium which is devoid of the above limitations.

U.S. Pat. Publ. No. 20050129663 teaches the use of dermis activated macrophages for axonal regeneration in the CNS, wound healing and treatment of myocardial infarction. However, this approach is technically laborious, necessitating the isolation of skin segments from the patient and co-incubation of same with the white blood cell sample. Additionally, the white blood cell sample may be subject to contamination arising from the skin segments and therefore cannot be favored as a drug. As expected, no clinical results using this approach for treating infarcted myocardium were reported.

GM-CSF stimulated macrophages were used for augmenting collateral vessel growth in an animal model of arteriogenesis (Herold 2004) but not for treating myocardial infarction which treatment requires also the formation of extracellular matrix in-order to regenerate a functional tissue.

The present inventors have previously used human activated macrophage suspensions for the treatment of unhealed ulcers and wounds by local injections of human activated macrophage suspension [Danon Exp Gerontol. 1997;32:633-41; Zuloff-Shani Transfus Apheresis Sci. 2004;30:163-7]. However, use of osmotically activated macrophages suspensions for the treatment of infarcted myocardium has never been suggested to date.

While reducing the present invention to practice, the present inventors have unexpectedly uncovered that myocardial infarction can be treated by administration of osmotically activated human immune cells into the infarcted myocardium thereby promoting angiogenesis, scar thickening and attenuating cardiac remodeling and dysfunction. These finding are of imperative clinical implications: myocardial infarction and heart failure are frequent in elderly people with impaired healing and regeneration capacity. Moreover, concomitant diseases such as diabetes and atherosclerosis have inhibitory effects on angiogenesis and healing. Thus, local delivery of activated immune cells, obtained from young donors, may provide an attractive alternative or adjunct cell-based therapy for myocardial infarction, particularly in elderly and sick patients. By promoting more effective tissue repair, it may be possible to reduce the deleterious remodeling, that is the leading cause of heart failure and death.

As is illustrated hereinbelow and in the Examples section which follows, human macrophage suspension was prepared from a whole blood unit obtained from young volunteer donors in a closed, sterile system and were activated by hypo-osmotic shock (see Example 1). Sprague-Dawley rats were subjected to extensive myocardial infarction (MI) and were immediately randomized to two injections of activated human macrophage suspension (2-4 ×10⁵ cells) or PBS (control) into the border of the infarcted myocardium. Viability and finctionality of the injected cells is described in Examples 2-3. Brirefly, hearts were harvested two months after injection for histological evaluation. Serial echocardiography studies, performed before and at two months after injection, showed that, compared with controls (n=9), macrophage implantation (n=8) improved scar thickness (0.87±0.02 vs. 0.98±0.04 cm; p<0.05), reduced left ventricular (LV) dilatation (LV diastolic dimension: 0.62±0.05 vs. 0.46±0.24 cm; p<0.05) and dysfunction ( LV fractional shortening: 20±4 vs.31±2%; p<0.05). Histological examination revealed that vessel density (/mm² ±SE) was significantly higher in macrophage injected hearts vs. control (25±4 vs. 10±1, p<0.05) and that the scar tissue of treated hearts was significantly populated with myofibroblasts.

Thus, the present invention shows, for the first time, that local injection of osmotically activated human macrophage suspension into the infarcted myocardium promotes angiogenesis, tissue repair and ameliorates cardiac remodeling and dysfunction.

Thus, according to one aspect of the present invention there is provided a method of treating an infarcted myocardium, the method comprising administering to the myocardium of a subject in need thereof a therapeutically effective amount of osmotically activated immune cells, thereby treating the infarcted myocardium.

As used herein the term “treating” refers to abrogating, substantially inhibiting, slowing or reversing the progression of a medical condition or disease associated with infarcted myocardium (e.g., acute MI) or substantially preventing the onset of myocardial infarction. Preferably, treating cures, e.g., substantially eliminates, the symptoms associated with an infarcted myocardium.

As used herein the phrase “infarcted myocardium” refers to a necrotic myocardial tissue caused by death of myocytes, coagulative necrosis, myocytolysis, contraction band necrosis, or apoptosis, resulting from a critical imbalance between the oxygen supply and demand of the myocardium.

Infarcted myocardium may be associated with (e.g., a consequence of-) numerous medical conditions and risk factors. Examples of such medical conditions include but are not limited to, atherosclerosis of the coronary arteries, left ventricular hypertrophy due to hypertension, viral disease, trauma, emboli to coronary arteries (e.g., due to cholesterol or infectious causes), coronary artery vasospasm, arteritis, coronary anomaly, drug abuse (e.g., cocaine, amphtamines and ephedrine). Risk factors for atherosclerotic plaque formation include, but are not limited to, age (e.g., male below 70), smoking, diabetes mellitus, hypercholesterolemia and hypertriglyceridemia, poorly controlled hypertension, family history and sedentary lifestyle.

As used herein the phrase “subject in need thereof” refers to a mammalian, preferably a human subject, who has been diagnosed with infarcted myocardium.

Methods of diagnosing an infarcted myocardium are well known in the art. Examples include, but are not limited to imaging methods such as echocardiography (e.g., use of 2-dimensional and M-mode echocardiography. when evaluating wall motion abnormalities and overall ventricular function. This assay can also identify complications of AMI); Technetium-99m sestamibi scan (Technetium-99m is a radioisotope that is taken up by the myocardium in proportion to the blood flow and is redistributed minimally after injection. This allows for time delay between injection and imaging. has potential use in identifying infarct in patients with atypical presentations or uninterpretable ECGs.) Thalium scan (Thallium accumulates in the viable myocardium) and cardiac magnetic resonance imaging (MRI) with or without contrast medium.

Myocardial infarction (e.g., AMI) may be diagnosed using various laboratory tests such as creatine kinase-MB, a standard for detecting myocardial necrosis, Myoglobin and/or Tropoinin I or T. The serum lactase dehydrogenase (LDH) level rises above the reference range within 24 hours of an AMI, reaches a peak within 3-6 days, and returns to. the baseline within 8-12 days.

Thus, as mentioned the subject in need thereof is administered with a therapeutically effective amount of osmotically activated immune cells.

As used herein the phrase “immune cells” refers to white blood cells which are derived from the bone marrow and are part of the immune system. Examples include the immune phagocytic system. Examples of immune phagocytic cells include, but are not limited to cells of the mononuclear phagocytic system, (MPS), including, but not limited to macrophages and monocytes. Other cells capable of phagocytosis include neutrophils, eosinophils and basophils.

Activated immune cells of the present invention are capable of secreting a cytokine repertoire which results in infarct healing. Without being bound by theory it is suggested that activated immune cells of the present invention play similar role as do the same cells at every stage of wound healing.(Cohen et al., 1987; Danon et al., 1989; Leibovich and Ross, 1975) In inflammation, macrophages have three major functions; antigen presentation, phagocytosis, and immunomodulation through production of various cytokines and growth factors.(Fujiwara and Kobayashi, 2005, Curr. Drug Targets Inflamm. Allergy 4:281-286). Macrophages play a critical role in the initiation, maintenance, and resolution of inflammation; inhibition of inflammation by removal or deactivation of mediators and inflammatory cells permit the host to repair damaged tissue.(Fujiwara and Kobayashi, 2005). Activated macrophages are deactivated by anti-inflammatory cytokines such as interleukin 10 and transforming growth factor β, and cytokine antagonists that are produced mainly by macrophages.(Thum et al., 2005) (Fujiwara and Kobayashi, 2005). In addition, macrophages control tissue vascularization after injury by releasing growth factors, matrix metalloproteinases (MMPs), and their inhibitors (Arras et al., 1998). Macrophages may also promote neovascularization directly by penetrating the extracellular matrix (Moldovan et al., 2000). Certain macrophage populations also have the potential to transform into vascular cells (Rehman et al., 2003). Thus, activated macrophages might modulate local inflammatory response, suppress local injury and promote tissue vascularization, healing and repair.

In animal models, depletion of macrophages using anti-macrophage serum showed impaired wound healing and decreased matrix production and fibrosis, indicating that macrophages are responsible for laying down matrix (Cohen et al., 1987; Leibovich and Ross, 1975).

Monocytes, macrophages and their cytokine products have been shown to accelerate vascularization in ischemic tissue,(exogenous GCSF expressing immune cells demonstrated by Herold et al., 2004) and promote infarct healing (Dewald et al., 2005 Circ. Res. 96:881-889 — showing the central effect of MCP-1 in infarct healing; Minatoguchi et al., 2004 Circulation 109:2572-2580, showing the effect of direct recombinant GCSF administration), myocyte protection,(Chazaud et al., 2003, J. Cell Biol. 163:1133-1143—showing the effect of satellite cell injection on monocyte attraction and muscle growth; Trial et al., 2004) and possibly regeneration.(Chazaud et al., 2003; Eisenberg et al., 2003; Minatoguchi et al., 2004). In a rat model of spinal injury, local implantation of macrophages that were pre-stimulated ex vivo resulted in nerve regeneration and partial functional recovery (Rapalino et al., 1998). These unique regenerative properties of macrophages could contribute to myocardial tissue repair and improve LV remodeling and function.

Immune cells of the present invention may be of an autologous or non-autologous (allogeneic or xenogeneic) origin. In fact since short-term function of the cells of the present invention is required (up to 7 days following administration, see Example 2), non-autologous cells may be preferred, as cells of young healthy donors may be used. Without being bound by theory, it is suggested that the use of non-autologous cells may even facilitate the healing process of the infarcted myocardium.

The following findings favor the use of non-autologous cells. Allogenic cells were found to be superior over autologous cells in rabbit model of hind limb ischemia (Herold et al., 2004). While transplantation of allogeneic cells resulted in a strong promotion of arteriogenesis, most likely through recruitment of recipient monocytes, transplantation of autologous cells (same animal) was not able to significantly augment collateralization (Herold et al., 2004). Indeed, recent observations have suggested that injection of human endothelial progenitor cells promote wound healing and vascularization by recruitment of local resident monocytes and macrophages (Suh et al., 2005, Stem Cells 23:1571-1578). In elderly and sick patients with impaired resident macrophage finction, injection of allogenic AMS obtained from young donors promotes wound healing and confirms the therapeutic effect of donor AMS.(Danon et al., 1997; Zuloff-Shani et al., 2004) Finally, the beneficial effect of allogenic cells can be explained by the modulation of local immune reactions in response to the transplanted cells.(Thum et al., 2005, J. Am. Coll. Cardiol. 46:1799-1802). It has been suggested that apoptotic cell ingestion by macrophages induces expression of anti-inflammatory cytokines that could suppress the damage of excessive inflammatory response (Thum et al., 2005).

Thus, white blood cell samples and preferably “peripheral blood mononuclear cells” (PBMCs) comprising a mixture of monocytes and lymphocytes are retrieved. Alternatively, cells are obtained from the blood bank. Several methods for isolating white blood cells are known in the art ( see e.g., Example 1). For example, PBMCs can be isolated from whole blood samples using density gradient centrifugation procedures. Typically, anticoagulated whole blood is layered over the separating medium. At the end of the centrifugation step, the following layers are visually observed from top to bottom: plasma/platelets, PBMCs, separating medium and erythrocytes/granulocytes. The PBMC layer is then removed and washed to remove contaminants prior to optional cell typing and cell viability assays.

A hermetically closed system for isolating white blood cell fraction from red blood cell fraction is taught in U.S. Pat. No. 6,146,890.

Regardless of the method employed, once the white blood cell sample is obtained cells are subjected to osmotic activation. It has also been found that this osmotic shock treatment of the monocytes in the white blood cells fraction enhances their differentiation into macrophages, as evidenced morphologically. Such a treatment may also be used to eliminate residual red blood cells which are more sensitive to changes in osmolarity than white blood cells.

As used herein the phrase “osmotically activated” refers to cell activation resultant of, preferably ex-vivo, incubation in hypo-osmotic (e.g., distilled water) or hyper-osmotic solution such as hypertonic saline.

Methods of osmotically activating cells of the present invention are described in Example 1 of the Examples section which follows as well as in U.S. Pat. No. 6,146,890.

Once obtained and iso-tonicity is re-established activated cells of the present invention may be typed and functionality (e.g., cytokine secretion) addressed using methods which are well known in the art. For example, monocyte activation may be confirmed by ELISA kits for interleukin levels in the supernatants obtained from the activated and non-activated monocyte cultures as described [Frenkel O., et al., Clin Exp Immunol 124:103-9 (2001)]. Phagocytosis capacity of fluorescent latex beads, by activated and non-activated monocytes collected from the culture bags was evaluated by FACS.

Various homogeneous and heterogeneous cell preparations may be obtained using the above teachings. One such functional cell preparation is described in Example 1 of the Examples section which follows.

Activated immune cells obtained as described herein are administered to the subject per se or in a pharmaceutical composition where they are mixed with suitable carriers or excipients.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

As used herein, the term “active ingredient” refers to the osmotically activated immune cells accountable for the intended biological effect.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa. which is herein fully incorporated by reference.

The pharmaceutical compositions of the present invention can be administered using any method known in the art. Preferably, the composition is administered in situ to the infarcated myocardial tissue. This can be effected by directly injecting the cells in or around the tissue region to be treated using a specially designed delivery catheters similar in principle to the perfusion catheters manufactured by Boston Scientific (USA) or via injection of the pharmaceutical composition directly into a tissue region of the subject. For example, mmyocardial cell implantation to a pre-specified region may be effected via intramyocardial injection, from either the epicardial or endocardial surface, or by intracoronary infusion. Other approaches include the use of devices that are designed to be placed retrogradely into the left ventricular chamber (endocardial approach) such as the Biosense-Webster Myostarm™ (Diamond Bar, Calif.), The Bioheart MyoCath™ (Bioheart, Inc., Santa Rosa, Calif.), and the Stiletto™ (Boston Scientific SciMed, Inc., Natick, Mass.). In addition, TransAccess (Medtronic) is a catheter-based system that allows direct myocardial access with IVUS-guided needle punctures through the coronary venous system and infusion catheter placement into remote myocardium. In addition, open chest surgery, Minimally Invasive Direct Coronary Artery Bypass (MID-CAB) surgery, or thorascopic surgery with direct exposure of the infarct (epicardial approach) are alternative efficient cell delivery approaches.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays as well as animal models. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Dosage amount and administration intervals may be adjusted individually to provide sufficient local concentration (i.e., minimally effective concentration, MEC). The MEC will vary for each preparation, but can be estimated from in vitro data. Dosages necessary to achieve the MEC will depend on individual characteristics, infarct size and route of administration. Based on animal studies it is estimated that 2-8×10⁶ cells will be required for 70-80 kg (˜1×10⁵/Kg body weight) adult with moderate infarction - again depending on mode of delivery. A proposed range of cells is 0.-10×10⁶ cells / Kg body weight.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

To improve therapeutic efficacy, compositions of the present invention may be administered along with well known therapeutic modalities for the treatment of infarcted myocardium (e.g., thrombolytic therapy).

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

Thus, the present invention provides methods which can be used to treat infarcted tissue.

The present invention is substantially less invasive than bypass surgery or angioplasty and as such it traverses the risks associated with such surgical techniques.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention dude molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Human Activated Macrophage Suspension (AMS) Tracking in the Infarcted Myocardium of Rats:

Human cells were detected in infarcted myocardium of rats using MRI, PCR and histological examination

Methods

Animal care—The study was performed in accordance with the guidelines of The Animal Care and Use Committee of Tel-Aviv University and Sheba Medical center - Israel, which conforms to the policies of the American Heart Association and the “Guide for the Care and Use of Laboratory Animals” (Department of Health and Human Services, NIH Publication no. 85-23). Overall, 44 rats were included in the study. The echocardiography functional study included 30 rats. Within 24 hours, nine rats died from the surgical procedure used to induce MI (four from AMS and five from the control group) and two rats, one from each group, died during follow-up. Thus, complete finctional analysis was performed in 17 rats treated with AMS (n=8) and saline (n=9). Another group of 16 rats was part of the cell tracking study by histology, PCR (n=8) and MRI (n=8).

Preparation of human activated macrophage suspension (AMS)—AMS was prepared as previously described [Danon D, et al., Exp Gerontol.32:633-41(1997); Zuloff-Shani A, et al. Transfus Apheresis Sci. 30:163-7 (2004)]. In brief, a whole blood unit donated routinely by healthy young donors (age range between 18-30 years) was collected into a triple blood bag system. The unit was then separated into packed red cells, white blood cells (buffy coat) and plasma. The bags containing the plasma and the buffy coat were connected, using a sterile connecting device (Terumo, Shizuoka, Japan), to the macrophage preparation system (Teva-Medical, Ashedod, Israel). Administration of CaCl₂ to the plasma bag induced coagulation. The serum thus obtained served as an autologous nutritional medium for the AMS. The buffy coat was treated by hypo-osmotic shock, and isotonicity was re-established following 45 seconds. Cells were then sedimented by centrifugation and the supernatant was transferred to an empty bag. Theosmotically shock-treated cells were resuspended in the donor serum and transferred into a culture bag containing sterile air. After incubation at 37° C., macrophages adherent to the bottom of the bag were collected and resuspended to a concentration of 2×10⁶ cells/ml. The total volume produced from one blood unit was 30-40 ml. Purity of cells was assessed by flow cytometry activated cell sorter (FACS) as described [Frenkel O., et al., Clin Exp Immunol 128:59-66 (2002)]. Monocyte activation was confirmed by ELISA kits for interleukin levels in the supernatants obtained from the activated and non-activated monocyte cultures as described [Frenkel O., et al., Clin Exp Immunol 124:103-9 (2001)]. Phagocytosis capacity of fluorescent latex beads, by activated and non-activated monocytes collected from the culture bags was evaluated by FACS.

Rat model of myocardial infarction (MI) and cell delivery—Male Sprague-Dawley female rats (˜250 g) were anesthetized with a combination of 90 mg/kg ketamine and 10 mg/kg xylazine, intubated and mechanically ventilated. The chest was opened by left thoracotomy, the pericardium was removed and the proximal left coronary artery was permanently occluded with an intramural stitch. The ischemic area was identified visually on the basis of pale color and segmental akinesis. One minute following coronary artery occlusion, rats were randomized to either two injections of 50 μL of AMS (total˜2-4×10P⁵ cells) or saline using a 27-gauge needle.

Cell labeling for In Vivo Tracking of AMS by MRI—To track the injected cells in vivo, AMS cells were labeled with a magnetic resonance (MR) contrast agent, TEndorem (Guerbet, Cedex France), an iron oxide nanoparticle solution provided with a total iron (Fe) content of 11.2 mg/ml and the transfection agent PLL (Poly-L-Lysine, catalogue No. P1524; Sigma, MW>388,000 and cell culture grade) as described [Frank JA, et al., Acad Radiol 9 Suppl 2:S484-7 (2002)]. The labeled and control cells were injected into the infarcted hearts (directly into the scar) of another group of rats (n=8) one minute after coronary artery ligation. From day one and every four days up to 14 days after cell delivery, the rat chest area was scanned using a 0.5T GE iMRI machine with a specially constructed animal probe. Imaging sequences included T1 spin echo and T2* gradient echo, as previously described (Barbash IM, 2004, Heart 90:87-91).

Cell tracking by PCR—Two, four, seven and 14 days after MI induction, DNA was extracted from rat heart treated with AMS or saline, and from human AMS (positive control) using QIAGEN's kit. The presence of human growth hormone (HGH) was determined by PCR amplification of a 434 bp fragment from the HGH gene (position 397-830 in GenBank Accession No. NG_{—}001334) using the following HGH sequence specific primers (SSP): forward: 5′ TGCCTTCCCAACCATTCCCTTA 3′ (SEQ ID NO: 1) and reverse 5′ CCACTCACGGATTTCTGTTGTGTTTC 3′ (SEQ ID NO: 2).

Histological Examination—Eight weeks following injection, animals were sacrificed with an overdose of phenobarbital. Hearts were sectioned into 4 transverse slices parallel to the atrioventricular ring. Each slice was fixed with 10% buffered formalin, embedded in paraffm, and sectioned with a microtome (5 μm thick). Serial sections were stained with Hematoxylin and Eosin and immunolabeled with antibodies against CD 68 —human monocyte and macrophage lineage antigen (Dako, Glostrup, Denmark), and HLA-DR (Dako, Glostrup, Denmark, data not shown). Neovascularization in the infarcted and peri-infarcted myocardium was assessed on representative slides obtained from mid-heart transverse section, immunostained with α-SMA antibodies (Sigma- Aldrich, St. Lewis, Mo, USA) to localize pericytes and arterioles. Five consecutive adjacent fields were photographed from each section at a magnification of 200 and the vessels were counted.

Resluts

AMS Characteristics—FACS analysis showed that AMS cell population contained 42.8% CD14 (a marker of human monocytes and macrophages) cells; 36.1% CD15 (a marker of myelomonocytic cells) cells; 0.02% CD34 (hematopoietic and endothelial progenitor cell marker) cells, and 21.1% CD19 (a marker of B cells) cells. By morphometric analysis, the suspension included monocytes (21%), segmental cells (51%), lymphocytes (21%), eosinophils (5%) and apoptotic cells (2%).

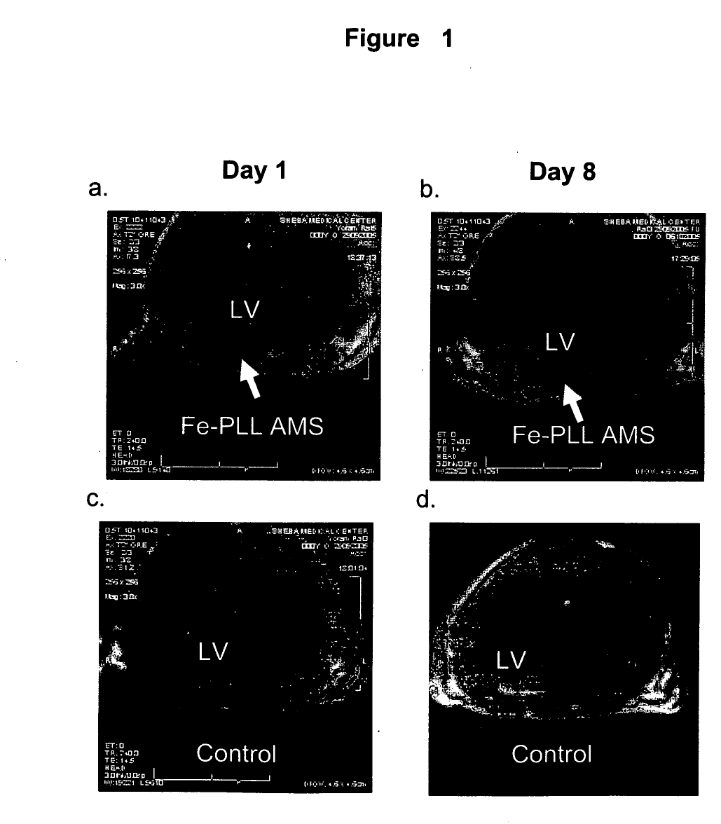

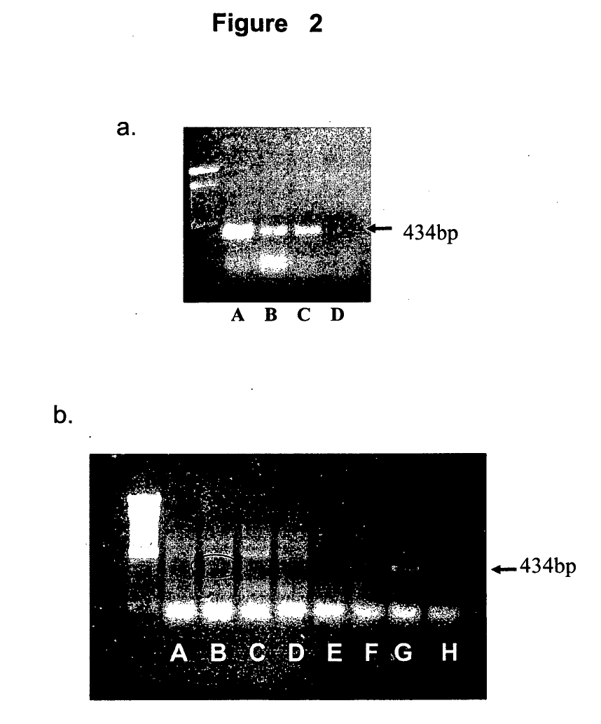

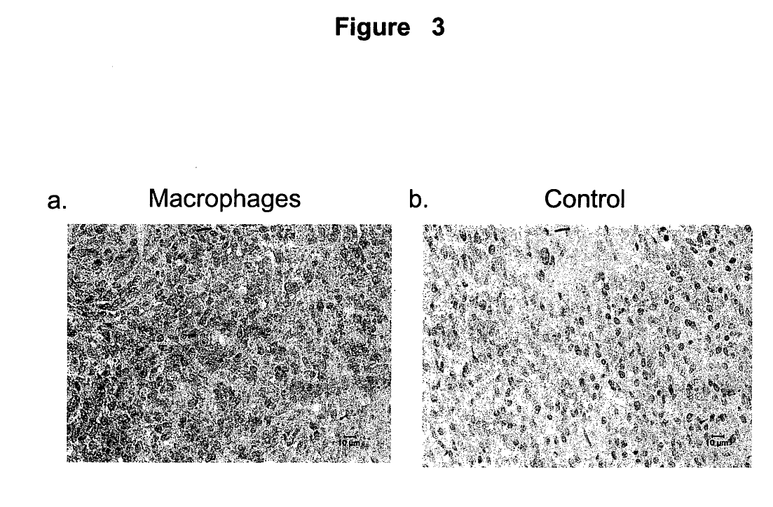

AMS tracking after injection—To track the injected cells in vivo, AMS cells were labeled with a magnetic resonance (MR) contrast agent, Endorem, prior to injection into infarcted myocardium of rats (n=8). Starting at day one and every four days up to14 days after cell delivery, the chest area was scanned using a 0.5T GE iMRI machine with a specially constructed animal probe and showed strong positive black signals from hearts treated with labeled AMS cells (FIGS. 1a-b) but not from non-labeled cells (FIGS. 1c-d). By conventional human GH gene specific PCR, human macrophages were detected in infarcted hearts (n=8). Strong PCR signals for human GH gene were found only in DNA preparations from two-day scars. Weak positive signals were observed in DNA from four- and seven-day scars treated by AMS but not in controls (FIGS. 2a-d). Two weeks after AMS injection, the PCR was negative in both AMS group and controls. By CD68 (a marker of human monocyte and macrophage) immunostaining, the injected human cells were identified in heart specimens obtained four days after injection (FIG. 3a). No staining was observed in control (saline) treated hearts (FIG. 3b) Human cells were not detected by CD68 and HLA-DR immunostaining in heart specimens obtained at one, two, four and eight weeks after injection (data not shown).

Table 1 below summarizes the results of the PCR. MRI and immuno-staining analysis. Some of the injected human cells survived at least 4 days (confirmed by 3 methods) and no more than 7 days. The persistent positive MRI signals may be related to iron nanoparticles released from dying labeled cells and engulfed by resident macrophages.

TABLE 1

| | Histology with | | |
|---|---|---|---|
| Day after | CD68 and HLADR | | MRI-iron labeled |
| injection | immunostaining | PCR for human GH | cells |
| Day 2 | Positive staining | Positive signal | Positive signal |
| Day 4 | Positive staining | Positive weak signal | Positive signal |
| Day 7-8 | Negative staining | Very weak signal | Positive signal |
| Day 14 | Negative staining | Negative signal | Positive signal |

Monocytes, macrophages and their cytokine products were shown to accelerate vascularization in ischemic tissue,(Herold et al., 2004) and promote infarct healing (Dewald et al., 2005; Minatoguchi et al., 2004), myocyte protection,(Chazaud et al., 2003; Trial et al., 2004) and regeneration.(Chazaud et al., 2003; Eisenberg et al., 2003; Minatoguchi et al., 2004). In a rat model of spinal injury, local implantation of macrophages that were pre-stimulated ex vivo resulted in nerve regeneration and partial functional recovery [Danon D, et al., J Wound Care.7:281-3 (1998)]. These unique regenerative properties of macrophages could contribute to myocardial tissue repair and improve LV remodeling and function. The results presented hereinbelow (Examples 2-3) shows AMS injection was associated with improved vascularization, myofibroblast accumulation, scar thickening and accumulation of resident macrophages which in turn, might contribute to infarct healing.[ Weber KT, et al., Clin Cardiol.19:447-55 (1996); Anversa P, et al., Circulation. 109:2832-8 (2004); Maekawa Y, et al., J Am Coll Cardiol.;44:1510-20 (2004)].

However, the role of macrophages in infarct repair is complex because macrophages produce a wide range of biologically active molecules participating in both beneficial and detrimental outcomes in inflammation. Macrophages could be beneficial in the early stage of infarct healing but deleterious during the late phase of scar formation and LV remodeling. Thus, control of macrophage activity by regulatory feedback is essential for effective healing and repair as well as avoiding excessive fibrosis. The present results show that the short life span of injected macrophages in the scar was ranged from four to seven days after injection, a life span which effectively improved healing and repair of the infarcted myocardium.

### Example 2

### Myocardial Repair in AMS Treated Infarcted Hearts

The onset of myocardial repair was assessed with immunostaining with EDI (a marker for rat tissue macrophages) and α-SMA (a marker for myofibroblast accumulation)

Methods

Preparation of human activated macrophage suspension (AMS)—preparation of AMS cells was effected as described in Example 1 above.

Rat model of myocardial infarction (MI) and cell delivery—myocardial infarction and cell delivery was effected as described in Example 1 above.

Histological Examination—histological sectioning was done as described in Example 1 above. Serial sections were immunolabeled with antibodies against α-smooth muscle actin (α-SMA) isoform (Sigma- Aldrich, St. Lewis, Mo., USA), and ED1 (Serotec, Raleigh, N.C. 27604, USA)—a marker for rat tissue-resident macrophages.

Results

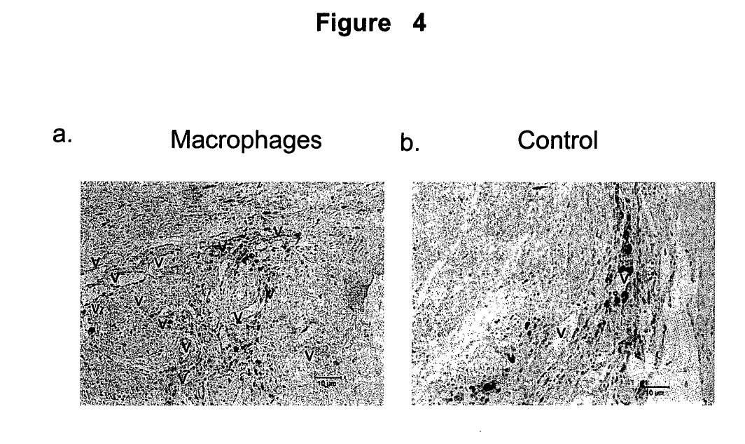

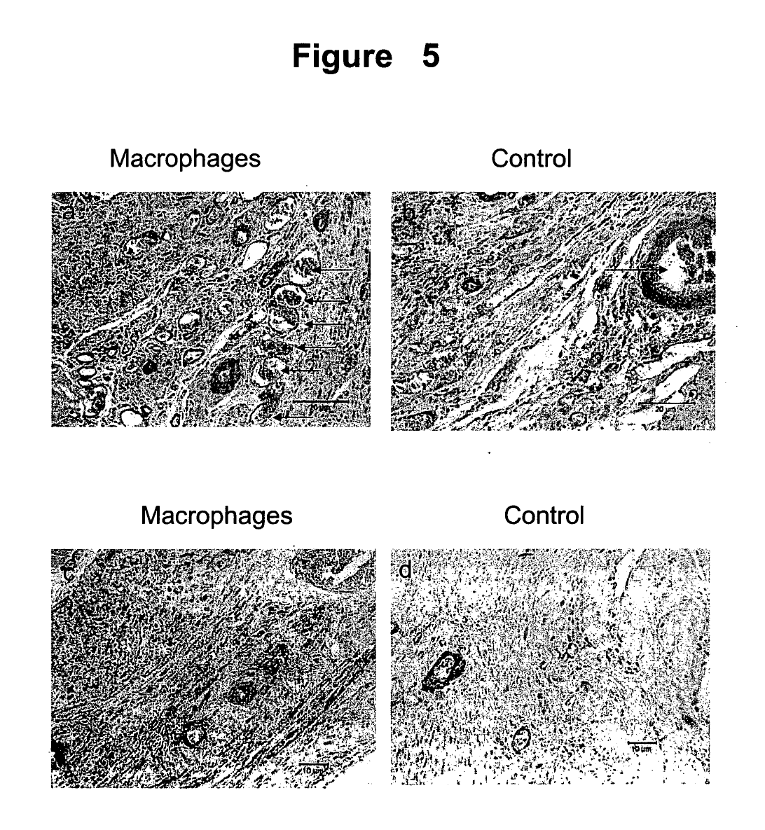

AMS Promotes Myocardial Repair—Two months after injection, immunostaining for ED1, a marker of rat tissue macrophages, revealed resident macrophage clusters associated with robust vascularization at the sites of AMS injections (FIG. 4a, brown clusters indicate macrophages). These results suggest that AMS transplantation promotes the recruitment of local monocytes, macrophages or both into the infarcted heart. Immunostaining for α-SMA, showed that AMS promoted myofibroblast accumulation and vascularization in the infarcted myocardium (FIGS. 5a,c) but not in control samples (FIGS. 5b, d).Vessel density in the scar tissue of AMS-treated animals was greater than controls (25±4 vs. 10±1/ mm²; p<0.05). These vessels were functional as indicated by red blood cells in the lumen (FIG. 5a). Myofibroblast accumulation in AMS-treated scars could contribute to scar contraction, thickening and strength. In control hearts, positive α-SMA staining was less extensive and mainly limited to the subendocardium and vessel walls.

Macrophages have been implicated in the pathogenesis of atherosclerosis and restenosis and injected AMS could theoretically accelerate atherosclerosis. However, it is suggested that by local delivery and targeting of macrophages into the necrotic tissue, vascular adverse effects can be avoided. The increase in tissue vascularization could be related to the inflammatory response and recruitment of resident macrophages triggered by the injection of human AMS into the rat heart. Indeed, recent observations have suggested that injection of human endothelial progenitor cells promote wound healing and vascularization by recruitment of local resident monocytes and macrophages [Maekawa Y, et al., J Am Coll Cardiol. 44:1510-20 (2004). Although the present study did not rule out such a possibility, it should be noted, however, that the healing capacity of macrophages has been proved in syngeneic animal models of wound healing [Frenkel O, et al., Clin Exp Immunol.;128:59-66 (2002)]. In addition, in elderly and sick patients with impaired resident macrophage function, injection of allogenic AMS promotes wound healing and confirms the therapeutic effect of donor AMS [Schaper J, et al., Virchows Arch A Pathol Anat Histol. 370:193-205 (1976); Polverini PJ, et al., Nature.269:804-6 (1977)]. Finally, the beneficial effect of allogenic cells can be explained by the modulation of local immune reactions in response to apoptosis of the transplanted cells. It has been suggested that apoptotic cell ingestion by macrophages induces expression of anti-inflammatory cytokines that could suppress the damage of excessive inflammatory response [Weber KT, et al., Clin Cardiol. 19:447-55 (1996)].

### Example 3

### Evaluation of Remodeling and Contractility of AMS Treated Hearts

AMS treated hearts were evaluated, compared to controls, for remodeling and contractility, using echocardiography

Methods

Preparation of human activated macrophage suspension (AMS)—preparation of AMS cells was done as described in Example 1.

Rat model of myocardial infarction (MI) and cell delivery—myocardial infarction and cell delivery was done as described in Example 1.

Echocardiography to Evaluate Remodeling and Contractility—Transthoracic echocardiography was performed on all animals within 24 hours after MI (baseline echocardiogram) and two months later. Echocardiograms were performed with a commercially available echocardiography system (Sonos 7500, Phillips, Andover, Mass., USA) equipped with 12-MHz phased-array transducer (Hewlett Packard, Palo Alto, Calif., USA). The following were measured, LV anterior wall thickness; maximal LV end-diastolic dimension; minimal LV end-systolic dimension and area in the short axis view by 2-D imaging; and fractional shortening (FS) as a measure of systolic function which was calculated as FS (%)=[(LVIDd-LVIDs)/LVIDd]×100, where LVID indicates LV internal dimension, s is systole, and d is diastole. All measurements were averaged over 3 consecutive cardiac cycles and were performed by an experienced technician who was blinded to the treatment group.

Statistical Analysis—Data are presented as means ± SE. Univariate differences between the control and treated groups were assessed with t test. Changes in echocardiography measurements and LV function between baseline and 8 weeks were assessed with paired t test using GraphPad Prism version 4.00 for Windows (GraphPad Software, San Diego, Calif., USA).

Results

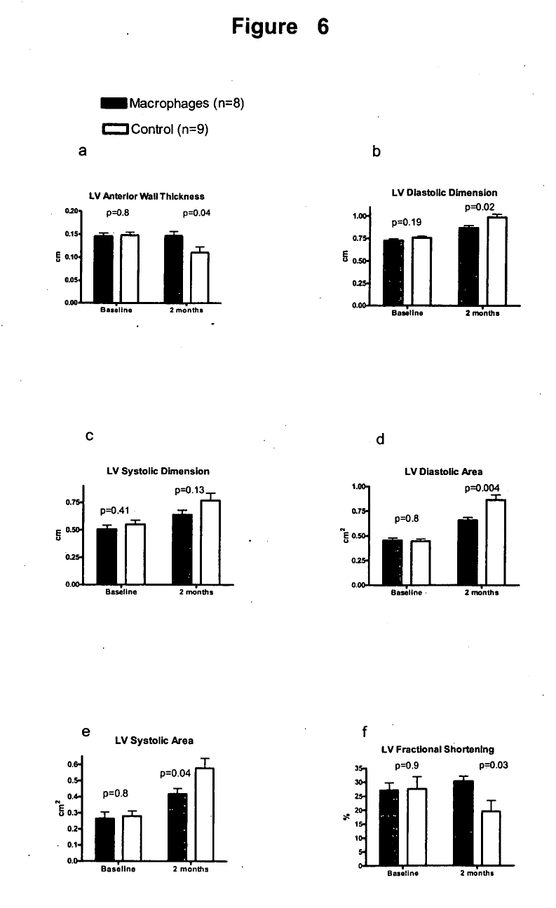

AMS Improves LV Remodeling and Function—Serial echocardiography studies, performed before and two months after injection, showed that AMS injection attenuated the typical course of LV remodeling, scar thinning and LV dysfunction (Table 2, FIG. 6). AMS significantly increased scar thickness (p<0.05; FIG. 6a), and reduced LV end diastolic (p<0.05; FIG. 6b), and end systolic dimensions (p=0.01; FIG. 6c), as compared with controls. Additionally, AMS diminished LV end diastolic (p<0.01; FIG. 6d) and end systolic areas (p<0.05; FIG. 6e), compared with controls. These favorable effects of AMS were associated with improved fractional shortening two months after MI (p<0.05; FIG. 6f).

TABLE 2

| | Activated macrophage | | | | | | |
|---|---|---|---|---|---|---|---|
| | suspension (n = 8) | | | Control (n = 9) | | | |
| | | | p | | | P | p |
| | | | baseline | | | baseline | Two months |
| | | Two | vs. two | | Two | vs. two | macrophages |
| | Baseline | months | months | Baseline | months | months | vs. control |
| AW d | 0.15 ± 0.01 | 0.15 ± 0.01 | 0.9 | 0.15 ± 0.02 | 0.11 ± 0.01 | 0.02 | <0.05 |
| cm* | | | | | | | |
| LVEDD | 0.72 ± 0.02 | 0.87 ± 0.03 | 0.001 | 0.75 ± 0.02 | 0.99 ± 0.04 | 0.0004 | <0.05 |
| cm† | | | | | | | |
| LVESD | 0.51 ± 0.04 | 0.64 ± 0.04 | 0.003 | 0.55 ± 0.04 | 0.77 ± 0.07 | 0.0008 | 0.01 |
| cm‡ | | | | | | | |
| LVEDA | 0.46 ± 0.02 | 0.66 ± 0.03 | <0.0001 | 0.45 ± 0.02 | 0.86 ± 0.05 | <0.0001 | <0.05 |
| cm²§ | | | | | | | |
| LVESA | 0.26 ± 0.04 | 0.42 ± 0.03 | 0.002 | 0.28 ± 0.03 | 0.58 ± 0.06 | 0.0004 | <0.05 |
| cm²\|\| | | | | | | | |
| LV SF | 27 ± 3 | 30 ± 5* | 0.19 | 27 ± 4 | 20 ± 4 | 0.06 | <0.05 |
| (%)# | | | | | | | |

*AW d = Anterior wall diastolic thickness;
†LVEDD = LV end diastolic dimension;
‡LVESD = LV end systolic dimension
§LV EDA = LV end diastolic area;
||LV ESA = LV end systolic area;
#LV FS = LV fractional shortening − [(LVIDd − LVIDs)/LVIDd] × 100.

The new results presented extend the data on the role of activated macrophages. in tissue healing. Results suggest that early after MI, injection of ex vivo activated AMS promotes myofibroblast accumulation, vascularization (as shown in Example 2) and scar thickening. The sum effect of scar thickening is reduction of wall stress (Laplace law), improved stabilization of chamber size, prevention of infarct expansion, and improved post MI function. Thus, a new approach for improving myocardial repair, particularly in elderly and sick patients can be sought after. In addition, the present findings challenge the dogma that inflammatory cells are always deleterious to the ischemic and infarcted myocardium. Inflammation and collagen synthesis are important steps that affect heart repair after MI [Frangogiannis NG, et al., Cardiovasc Res. 53:31-47 (2002)]. In fact, MI patients treated with anti-inflammatory drugs have experienced increased incidence of myocardial expansion, rupture and death [Zuloff-Shani A, et al., Transfus Apheresis Sci.30:163-167 (2004)].

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any. suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

### REFERENCES

1. Frangogiannis N G, Smith C W, Entman M L. The inflammatory response in myocardial infarction. Cardiovasc Res. 2002;53:31-47.

2. Sutton M G, Sharpe N. Left ventricular remodeling after myocardial infarction: pathophysiology and therapy. Circulation. 2000;101:2981-8.

3. Anversa P, Sussman M A, Bolli R. Molecular genetic advances in cardiovascular medicine: focus on the myocyte. Circulation. 2004;109:2832-8.

4. Forrester J S, Price M J, Makkar R R. Stem cell repair of infarcted myocardium: an overview for clinicians. Circulation. 2003;108:1139-45.

5. Lee M S, Makkar R R. Stem-cell transplantation in myocardial infarction: a status report. Ann Intern Med. 2004;140:729-37.

6. Edelberg J M, Xaymardan M, Rafii S, Hong M K. Adult cardiac stem cells—where do we go from here? Sci Aging Knowl Environ. 2003;2003:PE17.

7. Weber K T, Sun Y, Katwa L C. Wound healing following myocardial infarction. Clin Cardiol. 1996;19:447-55.

8. Sun Y, Kiani M F, Postlethwaite A E, Weber K T. Infarct scar as living tissue. Basic Res Cardiol. 2002;97:343-7.

9. Duffield J S. The inflammatory macrophage: a story of Jekyll and Hyde. Clin Sci (Lond). 2003;104:27-38.

10. Trial J, Rossen R D, Rubio J, Knowlton A A. Inflammation and Ischemia: Macrophages Activated by Fibronectin Fragments Enhance the Survival of Injured Cardiac Myocytes. Experimental Biology and Medicine. 2004;229:538-545.

11. Schaper J, Konig R, Franz D, Schaper W. The endothelial surface of growing coronary collateral arteries. Intimal margination and diapedesis of monocytes. A combined SEM and TEM study. Virchows Arch A Pathol Anat Histol. 1976;370: 193-205.

12. Polverini P J, Cotran P S, Gimbrone M A, Jr., Unanue E R. Activated macrophages induce vascular proliferation. Nature. 1977;269:804-6.

13. Moldovan N I, Goldschmidt-Clermont P J, Parker-Thomburg J, Shapiro S D, Kolattukudy P E. Contribution of Monocytes/Macrophages to Compensatory Neovascularization : The Drilling of Metalloelastase-Positive Tunnels in Ischemic Myocardium. Circ Res. 2000;87:378-384.

14. Rehman J, Li J, Orschell C M, March K L. Peripheral blood “endothelial progenitor cells” are derived from monocyte/macrophages and secrete angiogenic growth factors. Circulation. 2003;107:1164-9.

15. Eisenberg L M, Bums L, Eisenberg C A. Hematopoietic cells from bone marrow have the potential to differentiate into cardiomyocytes in vitro. Anat Rec A Discov Mol Cell Evol Biol. 2003;274:870-82.

16. Danon D, Madjar J, Edinov E, Knyszynski A, Brill S, Diamantshtein L, Shinar E. Treatment of human ulcers by application of macrophages prepared from a blood unit. Exp Gerontol. 1997;32:633-41.

17. Zuloff-Shani A, Kachel E, Frenkel O, Orenstein A, Shinar E, Danon D. Macrophage suspensions prepared from a blood unit for treatment of refractory human ulcers. Transfus Apheresis Sci. 2004;30: 163-7.

18. Frenkel O, Shani E, Ben-Bassat I, Brok-Simoni F, Shinar E, Danon D. Activation of human monocytes/macrophages by hypo-osmotic shock. Clin Exp Immunol. 2001;124:103-9.

19. Frenkel O, Shani E, Ben-Bassat I, Brok-Simoni F, Rozenfeld-Granot G, Kajakaro G, Rechavi G, Amariglio N, Shinar E, Danon D. Activated macrophages for treating skin ulceration: gene expression in human monocytes after hypo-osmotic shock. Clin Exp Immunol. 2002;128:59-66.

20. Leor J, Patterson M, Quinones M J, Kedes L H, Kloner R A. Transplantation of fetal myocardial tissue into the infarcted myocardium of rat. A potential method for repair of infarcted myocardium? Circulation. 1996;94:II332-6.

21. Ando H, Kubin T, Schaper W, Schaper J. Cardiac microvascular endothelial cells express alpha -smooth muscle actin and show low NOS III activity. Am J Physiol Heart Circ Physiol. 1999;276:H1755-1768.

22. Etzion S, Battler A, Barbash I M, Cagnano E, Zarin P, Granot Y. Kedes L H, Kloner R A, Leor J. Influence of embryonic cardiomyocyte transplantation on the progression of heart failure in a rat model of extensive myocardial infarction. J Mol Cell Cardiol. 2001;33:1321-30.

23. Leor J, Aboulafia-Etzion S, Dar A, Shapiro L, Barbash I M, Battler A, Granot Y, Cohen S. Bioengineered cardiac grafts: A new approach to repair the infarcted myocardium? Circulation. 2000;102:III56-61.

24. Danon D, Frenkel O, Diamandstein L, Winkler E, Orenstein A. Macrophage treatment of pressure sores in paraplegia. J Wound Care. 1998;7:281-3.

25. Maekawa Y, Anzai T, Yoshikawa T, Sugano Y, Mahara K, Kohno T, Takahashi T, Ogawa S. Effect of granulocyte-macrophage colony-stimulating factor inducer on left ventricular remodeling after acute myocardial infarction. J Am Coll Cardiol. 2004;44:1510-20.

26. Hammerman H, Kloner R A, Schoen F J, Brown E J, Jr., Hale S, Braunwald E. Indomethacin-induced scar thinning after experimental myocardial infarction. Circulation. 1983;67:1290-5.

27. Roberts R, DeMello V, Sobel B E. Deleterious effects of methylprednisolone in patients with myocardial infarction. Circulation. 1976;53:1204-6.

28. Monaco J L, Lawrence W T. Acute wound healing an overview. Clin Plast Surg. 2003;30:1-12.

29. Park J E, Barbul A. Understanding the role of immune regulation in wound healing. Am J Surg. 2004;187:11S-16S.

30. Cohen B J, Danon D, Roth G S. Wound repair in mice as influenced by age and antimacrophage serum. J Gerontol. 1987;42:295-301.

31. Leibovich S J, Ross R. The role of the macrophage in wound repair. A study with hydrocortisone and antimacrophage serum. Am J Pathol. 1975;78:71-100.

32. Singer A J, Clark R A. Cutaneous wound healing. N Engl J Med. 1999;341 :738-46.

33. DiPietro L A. Wound healing: the role of the macrophage and other immune cells. Shock. 1995;4:233-40.

34. Witte M B, Barbul A. General principles of wound healing. Surg Clin North Am. 1997;77:509-28.

35. Johnson C, Sung H J, Lessner S M, Fini M E, Galis Z S. Matrix metalloproteinase-9 is required for adequate angiogenic revascularization of ischemic tissues: potential role in capillary branching. Circ Res. 2004;94:262-8.

36. Arras M, Ito W D, Scholz D, Winkler B, Schaper J, Schaper W. Monocyte activation in angiogenesis and collateral growth in the rabbit hindlimb. J Clin Invest. 1998;101:40-50.

37. Heil M, Schaper W. Influence of mechanical, cellular, and molecular factors on collateral artery growth (arteriogeriesis). Circ Res. 2004;95:449-58.

38. Weihrauch D, Arras M, Zimmermann R, Schaper J. Importance of monocytes/macrophages and fibroblasts for healing of micronecroses in porcine myocardium. Mol Cell Biochem. 1995;147:13-9.

39. Ito W D, Arras M, Winkler B, Scholz D, Schaper J, Schaper W. Monocyte chemotactic protein-1 increases collateral and peripheral conductance after femoral artery occlusion. Circ Res. 1997;80:829-37.

40. Buschmann I R, Hoefer I E, van Royen N, Katzer E, Braun-Dulleaus R, Heil M, Kostin S, Bode C, Schaper W. G M-C S F: a strong arteriogenic factor acting by amplification of monocyte finction. Atherosclerosis. 2001;159:343-56.

41. Iba O, Matsubara H, Nozawa Y, Fujiyama S, Amano K, Mori Y, Kojima H, Iwasaka T. Angiogenesis by implantation of peripheral blood mononuclear cells and platelets into ischemic limbs. Circulation. 2002;106:2019-25.

42. Heil M, Ziegelhoeffer T, Pipp F, Kostin S, Martin S, Clauss M, Schaper W. Blood monocyte concentration is critical for enhancement of collateral artery growth. Am J Physiol Heart Circ Physiol. 2002;283:H2411-9.

43. Herold J, Pipp F, Fernandez B, Xing Z, Heil M, Tillmanns H, Braun-Dullaeus R C. Transplantation of monocytes: a novel strategy for in vivo augmentation of collateral vessel growth. Hum Gene Ther. 2004;15:1-12.

44. Urbich C, Heeschen C, Aicher A, Dembach E, Zeiher A M, Dimmeler S. Relevance of monocytic features for neovascularization capacity of circulating endothelial progenitor cells. Circulation. 2003;108:2511-6.

45. Perin E C, Dohmann H F R, Borojevic R, Silva S A, Sousa A L S, Silva G V, Mesquita C T, Belem L, Vaughn W K, Rangel F O D, Assad J A R, Carvalho A C, Branco R V C, Rossi M I D, Dohmann H J F, Willerson J T. Improved Exercise Capacity and Ischemia 6 and 12 Months After Transendocardial Injection of Autologous Bone Marrow Mononuclear Cells for Ischemic Cardiomyopathy. Circulation. 2004;110:II-213-218.

46. Gulati R, Jevremovic D, Peterson T E, Chatterjee S, Shah V, Vile R G, Simari R D. Diverse Origin and Function of Cells With Endothelial Phenotype Obtained From Adult Human Blood. Circ Res. 2003;93:1023-1025.

47. Rapalino O, Lazarov-Spiegler O, Agranov E, Velan G J, Yoles E, Fraidakis M, Solomon A, Gepstein R, Katz A, Belkin M, Hadani M, Schwartz M. Implantation of stimulated homologous macrophages results in partial recovery of paraplegic rats. Nat Med. 1998;4:814-21.

48. Muller-Ehmsen J, Peterson K L, Kedes L, Whittaker P, Dow J S, Long T I, Laird P W, Kloner R A. Rebuilding a damaged heart: long-term survival of transplanted neonatal rat cardiomyocytes after myocardial infarction and effect on cardiac function. Circulation. 2002;105:1720-6.

49. Danon D, Kowatch M A, Roth G S. Promotion of wound repair in old mice by local injection of macrophages. Proc Natl Acad Sci USA. 1989;86:2018-20.

50. Vasa M, Fichtlscherer S, Aicher A, Adler K, Urbich C, Martin H, Zeiher A M, Dimmeler S. Number and migratory activity of circulating endothelial progenitor cells inversely correlate with risk factors for coronary artery disease. Circ Res. 2001;89:E1-7.

51. Waltenberger J. Impaired collateral vessel development in diabetes: potential cellular mechanisms and therapeutic implications. Cardiovasc Res. 2001 ;49:554-60.

What is claimed is:

1. A method of treating an infarcted myocardium, the method comprising administering to the myocardium of a subject in need thereof a therapeutically effective amount of osmotically activated immune cells, thereby treating the infarcted myocardium.

2. The method of claim 1, wherein said osmotically activated immune cells are ex-vivo activated.

3. The method of claim 1, wherein said osmotically activated immune cells comprise hypo-osmotically activated immune cells.

4. The method of claim 1, said administering comprises local administering.

5. The method of claim 4, wherein said local administering is effected by injection.

6. The method of claim 1, wherein the infarcted myocardium is associated with a disease or condition selected from the group consisting of atherosclerosis, ventricular hypertrophy, hypoxia, emboli to coronary arteries, coronary artery vasospasm, arteritis, coronary anomaly.

7. The method of claim 1, further comprising activating white blood cells so as to obtain said osmotically activated immune cells prior to said administering.

8. The method of claim 5, wherein said osmotically activated immune cells comprise immune phagocytic cells.

9. The method of claim 8, wherein said immune phagocytic cells comprise macrophages.

10. The method of claim 7, wherein said activating is effected by subjecting said white blood cells to a hypotonic solution.

11. The method of claim 10, wherein said hypotonic solution is distilled water.

12. The method of claim 1, wherein said osmotically activated immune cells comprise non-autologous cells.

13. The method of claim 12, wherein said non-autologous cells comprise xenogeneic cells.

14. The method of claim 12, wherein said non-autologous cells comprise allogeneic cells.

15. The method of claim 1, wherein said osmotically activated immune cells are administered at an amount selected from 0.1 -10×10⁶ cells/Kg body weight.

16. An article of manufacturing comprising packaging material and a pharmaceutical composition identified for treating an infarcted myocardium being contained within the packaging material, the pharmaceutical composition comprising, as an active ingredient, osmotically activated immune cells and a pharmaceutically acceptable carrier.

17. The article of manufacturing of claim 16, said pharmaceutical composition is formulated for local administration.

18. The article of manufacturing of claim 16, wherein said infarcted myocardium is associated with a disease or condition selected from the group consisting of atherosclerosis, ventricular hypertrophy, hypoxia, emboli to coronary arteries, coronary artery vasospasm, arteritis, coronary anomaly.

19. The article of manufacturing of claim 16, wherein said osmotically activated immune cells comprise macrophages.

20. The article of manufacturing of claim 16, wherein said osmotically activated immune cells comprise non-autologous cells.

21. The article of manufacturing of claim 20, wherein said non-autologous cells comprise xenogeneic cells.

22. The article of manufacturing of claim 20, wherein said non-autologous cells comprise allogeneic cells.

23. The article of manufacturing of claim 16, wherein said osmotically activated immune cells comprise hypo-osmotically activated immune cells.

24. The article of manufacturing of claim 16, wherein said osmotically activated immune cells are ex-vivo activated.


SEQUENCE LISTING (2 sequences)

SEQ ID NO: 1 - DNA, 22 bp, Artificial sequence
  Single strand DNA oligonucleotide
tgccttcccaaccattccctta

SEQ ID NO: 2 - DNA, 26 bp, Artificial sequence
  Single strand DNA oligonucleotide
ccactcacggatttctgttgtgtttc